SEQUENCE LISTING (35 sequences)

SEQ ID NO: 1 - DNA, 918 bp, Pinus sylvestris
ctgaaggagtagaaaatctactgcgttctctgcatttgttgtttattatatcgtccgcaatttttacgaagttatttactcgtcttagtgttttaaattagtattaagatggcggaagaagcaccagagcacaaggaccgcggtatgttcggcttgttcggcaaaaagaaggaagatgagggaaagcaaagtgatcacgttgtgcagactcctgctcggactgaggctgcctcttattatccacctgctcctcagcatggagtcgaacaaggccacggtcatggtcacgaggggcagtttgcccgtgaggaagctgagcaacagaaacacacgggaaagcttcaccgctcgaacagttccagctccagctcttcgagcgatgaagaggaggaaggcagaaagaagaaagaaggcggaaggaagaagaaagggtcaaaggacaagaccaaggaaaagctccccggggacggaaagcagtatcccggtgagcatgaaaagcagtattcccgtcagcatgggggtgaagaggaaaagaaggcgggtatgcttgataaaatcaaagcgaagctgccgggacaacataacaagaatgatggggaacagtaagagaagaagcaccccctgcccatataatatgtgaatctgtgtctactcgttcagggcgttgcttaggagcatgcatggatgagaataatggtacgtgcgtgttgtggggttttgattagtaatgtgttgtgttacaagaggggataatgtaatccgatcaatccgcattgccattggtgatgtcggaatttgtggttttataataaatggggtttcggtacgagcttaccttcagcgtctgtccgatgtatcgattaaagttatgaatatattataatcagtattttcatgtgaaaaaaaaaaaaaaaaaaa

SEQ ID NO: 2 - DNA, 647 bp, Pinus sylvestris
aatttgttgtttgttatatcgtctgcaatttttaccaagttttttactcgttattaagatggcggaagaacaacaggaccgcggtatgttcggcttattgggcaaaaagaaggaagatgagggaaagcaatgtgatcagactgagggtggtagatgtgaagaggggaagaagccgggaatggtagataaaatcaaagagaagattccgggacaacagcaaaagctccccggagacggaaagcagtgtggcggtcagcctgggcgtgaagaggaaaagaagccaggtatggttgataaaatcaaacagaagctgccgggacaacaaaataagaatgacgtggagtaagagaagatagtgtaaagaagaccccctgcccatctaatatgtgaatctgtgtacactctcgttcagggcattgcttaggagcatgcatggatgagaataatgttatgtgcgtgttgattagtaatgtgttgtgttacaagagggggatcaaagttacgactgtaatccgatggtgaggtcggtatttgcggttttataataaatagggtttcgagtaccatcttttacctgagcgtgtttccgatttatcggttgaagttatgaatatagtttatgtgaccaaactttcagcaatgttcaa

SEQ ID NO: 3 - DNA, 681 bp, Pinus sylvestris
  misc_feature (112)..(112): wherein n is a, c, g, or t
tgttttctctaacatttgtgattcaagcctgtgcgtggggtattcgaatccagtcgccgcatatatagtcggtgtgtctttttgtgtgtataagcatgtctgagggacagcngtcaccacctcttcaaccgcaaaaaggaggaggacaacgttaattctggctctgctgaccagggctcctatgggtcctctgattaccaggctggctctggtaataacgctggctctggttataacgccgacactggttataaggccgattacggcaatgatggtcagcaaggtcaatatgatggtcagcaaggtcaatcacagaactatgggggagcccaaaaggaggagaactatgggggagcccaaaaggaagtgaaacaagaccagcgcaaggagcgcttcggggagctcggaaccgcggctgctggaggctatgcactgtatgagaggaatgaggcaaaaaaggatcctgagaatgctaggaggcacaagacagaggaggaggtcgctgctgcgggcgctgtgggcaccggtgggtacgcgttccacgagcgccacgagaagaaacgagacgaggaaaccgaggaagccgagggtggccgcaagcctcgccacaacctcttctgaatttggccatgcctctctaatggtggacttttggagcagaagccaattatagcagttggc

SEQ ID NO: 4 - DNA, 590 bp, Pinus sylvestris
  misc_feature (115)..(115): wherein n is a, c, g, or t
cgaatacagcagtaacaggcgtggtggtattgcaggaggcgtctttgcaaacgccttcttcatccgatttcctacgttcgccgtttcttcgcagtttttctcttttttttttttnagggtttnttttccttcgncnccagcnaaaangananagngcntntcnatccanatnggacaggccggtntccaggtngnaaatgcttgctggnaactttactgcctcnancacggnnttnancctganggccaaatncccagcnacaaaactgnngggggnggaaacaatgcnttnaacanttttttnancaaaactgganccgnaaagcntgtnccccncnctgtntttgnaaatntggacccnactgtcnttgacaaggtacggactggtncttaccgtnagnttttccatcctnaacagnttatnagcggcaaggaaaatgcggccaanaactttgcccgnggtcnttacnccattggcaaggaaattggggacctntgcctggaccgtattcgcaagctggctgacaactgcncgggtcttnaaggattcctcgtnttcaacncagttgggggaggaactggttctggtc

SEQ ID NO: 5 - DNA, 537 bp, Pinus sylvestris
aatttgttgtttgttatatcgtctgcaatttttactaagttttttactcgttattaagatggcgcaaccagcacaccaggaccgcggtatgttcggcaaaaagaaggaagatgagggaaagcaatgtgatcagactgagggtggtagatgtgaagaggggaagaaaccgggaatggtagataaaatcaaagagaagattccgggacaacagcaaaagctccccggagacggaaagcagtgttgcggtcagcctggaggtgaagaggaaaagaagccaggtatggttgataaaatcaaacagaagctgccgggacaacaaaacaagactggttgtgattaagagaagatagtgtagtctgtgtttactcctccagagcttgggtgaggagcatggatgagaataatcgtatgtgcgtttgtggtattgtaataaatgggacttcgggtacaatcgtacctgagcgtctgtccgatgtatcgattaaagttatgaatatattataatagttttttcatattaaaaaaaaaaaaaaaaaa

SEQ ID NO: 6 - DNA, 337 bp, Pinus sylvestris
aagatggcgcaaccagcacaccaggaccgcggtatgttcggcaaaaagaaggaagatgagggaaggcaatgtgatcagactgagggtggtagatgtgaagaggggaagaagccgggaatggtagataaaatcaaagagaagattccgggacaacagcaaaagctccccggagacggaaagcagtgtggcggtcagcctgggcgtgaagaggaaaagaagccaggtatggttgataaaatcaaacagaagctgccgggacaacaaaataagaatgacgtggagtaagagaagatagtgtaaagaagaccccctgcccatctaatatgtgaatctgtgt

SEQ ID NO: 7 - DNA, 409 bp, Pinus sylvestris
gggctatttgctgtttgttatatcgtctgcagtttttaccaacttttttactcgttattaagatggcggaagaacaacacgaccgcggtatgttcggcttattgtgcaaaaagaaggaagatgagggaaagcaatgtgatcacactgagggtggtagatgtgaagaggggaagaagccgggaatggtcgataaaatcaaagagaagattctgggacaacagccaaagcttccccgagaccgaaagcagtgtggctgtcaccctggtcgtgtagaggaaaagagaccacgtatggttgataaaatcaaacagaagctgccgggacatcaaaataaaaatgacgtggagtaagagaagatattgtttagaagacccccctgccctctcatatgtgaatatgtgtacactct

SEQ ID NO: 8 - DNA, 559 bp, Pinus sylvestris
gcgttctgtcaatttgttgtttgttatatcgtctgcattttttaccaagttttttactcgttattaagatggcggaagagcaacaggaccgcggtatgttcggcttattgggcaaaaagaaggaagatgagggaaagcaatgtgatcagactgagggtggtagatgtgaagaggggaagaagccgggaatggtagataaagtcaaagagaagattccgggacaacagcaaaagctccccggagacggaaagcagtgttgcggtcagcctggaggtgaagaggaaaagaagccaggtatggttgataaaatcaaacagaagctgccgggacaacaaaacaagactggttgtgattaagagaagatagtgtagtctgtgtttactcctccagagcttgggtgaggagcatggatgagaataatagtatgtgcgtttgtgtttggggtattataataaatgggacttcgggtacaagcgtacctgagcgtctgtccgatgtatcaattaaagttatgaatatactataaaagttttttcatgttaaaaaaaaaaaaaaaaaa

SEQ ID NO: 9 - DNA, 341 bp, Pinus sylvestris
atttgttgtttgttatatcgtctgcatttttcaccaaagttatttactcgttattaagatggcggaagagcaacaggaccgcggtatgttcggcttattaggcaaaaagaaggaagatgagggaaagcaatgtgatcagggtcctcctcggactgaggttggtagatgtgaagaggggaagaagccgggaatggtagataaaatcaaagagaagattccggggcaacagcaaaagctccccggagacggaaagcagtgtggcggtcagcctgggcgtgaagaggaaaagaagccaggtatggttgataaaatcaaacagaagctgccgggacaacaaaata

SEQ ID NO: 10 - DNA, 1143 bp, Pinus sylvestris
catcacccaaagtgaggctcagaatgaggaggctcagaatgaggcttgcttcttattatccagcttgctcctccgcatggagccgaacacggcccatggtcacgaggggcaactttacccctgaggaagctgaggaaaagaaacacacgggtttgataggaaagcttcaccgcacccacagttccagctccagctcttctagtgatgaagaggaagaaggcgagaaaaagaaagagaaagagagaaagaagaaagaggcaaaggacaagaccaataaaaagctacctggagatggacagcattcttcagatcagtctgggattgaagaagagaagaaagtaagtttggttgataaattcaaagagaagctccctgcacaacctaacaagggggagggggaggagaaagtggaagtggagaagaaagcaggtgtcaaagaaaagctccctgtacaacctaacaagggggaaagggaagagaaagcggaagtggagaagaaagtaggtttgctagataaaattaaagagaagctacccggacactctaaccaaaaaggaaggggaagggaaaagaagaagaagaagcagaagtggcgaaaaaggcgagtcctgatcgataaaatcaaagagaaactccccggacatcataccaagaaggaaggagaagaggaagaaaagaagcaaaactattaaagctactgtaaacaagatcttgtccaagtctatatttaactcgttccttactttgcctgagagcattgataggaatgatgatatgtatctgttgtgtggttttgattaatgatttgcgttagaagagaagataaaagttatgaatgtgagccgacggatccacattggcatctgcagtattgcatatttcatgaacggggttccaattaccttacccgggtcctgagtgttcggtcaagttattatcatatatttatggacatttttatatgtggcaacaaggattgcttgggatccaggaagtaagcaggatttcattaatcatgaattgtatctggccttttgagttgcaatatcttttggctggaattggcaacttcttcctgagtgtggtgttgtagttatgtatgagaggatcatatggatatgtaaagtccgtcataaaacttaa

SEQ ID NO: 11 - protein, 222 aa, Pinus sylvestris
MRRLRMRLASYYPACSSAWSRTRPMVTRGNFTPEEAEEKKHTGLIGKLHRTHSSSSSSSSDEEEEGEKKKEKERKKKEAKDKTNKKLPGDGQHSSDQSGIEEEKKVSLVDKFKEKLPAQPNKGEGEEKVEVEKKAGVKEKLPVQPNKGEREEKAEVEKKVGLLDKIKEKLPGHSNQKGRGREKKKKKQKWRKRRVLIDKIKEKLPGHHTKKEGEEEEKKQNY

SEQ ID NO: 12 - protein, 164 aa, Pinus sylvestris
MAEEAPEHKDRGMFGLFGKKKEDEGKQSDHVVQTPARTEAASYYPPAPQHGVEQGHGHGHEGQFAREEAEQQKHTGKLHRSNSSSSSSSSDEEEEGRKKKEGGRKKKGSKDKTKEKLPGDGKQYPGEHEKQYSRQHGGEEEKKAGMLDKIKAKLPGQNKNDGEQ

SEQ ID NO: 13 - protein, 93 aa, Pinus sylvestris
MAQPAHQDRGMFGKKKEDEGKQCDQTEGGRCEEGKKPGMVDKIKEKIPGQQQKLPGDGKQCCGQPGGEEEKKPGMVDKIKQKLPGQQNKTGCD

SEQ ID NO: 14 - protein, 93 aa, Pinus sylvestris
MAQPAHQDRGMFGKKKEDEGRQCDQTEGGRCEEGKKPGMVDKIKEKIPGQQQKLPGDGKQCGGQPGREEEKKPGMVDKIKQKLPGQQNKNDVE

SEQ ID NO: 15 - protein, 95 aa, Pinus sylvestris
MAEEQQDRGMFGLLGKKKEDEGKQCDQTEGGRCEEGKKPGMVDKIKEKIPGQQQKLPGDGKQCGGQPGREEEKKPGMVDKIKQKLPGQQNKNDVE

SEQ ID NO: 16 - protein, 95 aa, Pinus sylvestris
MAEEQHDRGMFGLLCKKKEDEGKQCDHTEGGRCEEGKKPGMVDKIKEKILGQQPKLPRDRKQCGCHPGRVEEKRPRMVDKIKQKLPGHQNKNDVE

SEQ ID NO: 17 - protein, 95 aa, Pinus sylvestris
MAEEQQDRGMFGLLGKKKEDEGKQCDQTEGGRCEEGKKPGMVDKVKEKIPGQQQKLPGDGKQCCGQPGGEEEKKPGMVDKIKQKLPGQQNKTGCD

SEQ ID NO: 18 - protein, 94 aa, Pinus sylvestris
MAEEQQDRGMFGLLGKKKEDEGKQCDQGPPRTEVGRCEEGKKPGMVDKIKEKIPGQQQKLPGDGKQCGGQPGREEEKKPGMVDKIKQKLPGQQN

SEQ ID NO: 19 - protein, 8 aa, Unknown
  plant
KIKEKLPG

SEQ ID NO: 20 - protein, 8 aa, Unknown
  plant
SSSSSSSS

SEQ ID NO: 21 - protein, 6 aa, Unknown
  plant
DEYGNP

SEQ ID NO: 22 - DNA, 20 bp, Unknown
  plant
tgacggagaattagggttcg

SEQ ID NO: 23 - DNA, 20 bp, Unknown
  plant
cctccaatggatcctcgtta

SEQ ID NO: 24 - protein, 15 aa, Unknown
  plant
GREKKKKKQKWRKRR

SEQ ID NO: 25 - DNA, 17 bp, Unknown
  plant
cacacgggtttgatagg

SEQ ID NO: 26 - DNA, 20 bp, Unknown
  plant
tgatctgaagaatgctgtcc

SEQ ID NO: 27 - DNA, 25 bp, Unknown
  plant
tgagaataatggtacgtgcgtgttg

SEQ ID NO: 28 - DNA, 25 bp, Unknown
  plant
ctgaaggtaagctcgtaccgaaacc

SEQ ID NO: 29 - DNA, 21 bp, Unknown
  plant
gttcagggcattgcttaggag

SEQ ID NO: 30 - DNA, 21 bp, Unknown
  plant
gcaaataccgacctcaccatc

SEQ ID NO: 31 - DNA, 18 bp, Unknown
  plant
ggaagaaaccgggaatgg

SEQ ID NO: 32 - DNA, 18 bp, Unknown
  plant
ttttgttgtcccggcagc

SEQ ID NO: 33 - protein, 245 aa, Picea glauca
MAEQAPEHQDRGMFGLFGKKKEEEGRQDDQISQTPATHTHNEAQTEAASYYPTSPHEVKHGPGHGHEGQLTPEEAEQQKHKGLVGKLHRTHSSSSSSSSDEEEEGGKKKEVGRKKKGAKDKTKEKLPSGGHHSSDHCGGEEKKKAGMVDKIKEKLPGHQEKLPAGGEHSSDQCGTKEEKKTGLLDKIKDKLPGHQEKLPGGGQHSSDQYGGKQEKKMGLLDKIKEKLPGHENKNDGEEEEKKHPH

SEQ ID NO: 34 - protein, 9 aa, Unknown
  plant
DKXKXKLPG

SEQ ID NO: 35 - protein, 16 aa, Unknown
  plant
EKKPGMVDKIKXKLPG

(19) United States
(12) Patent Application Publication
(10) Pub. No.: US 2015/0376720 A1
(43) Pub. Date: Dec. 31, 2015

(54) METHODS AND MEANS FOR DETERMINING AND CONFERRING STRESS TOLERANCE IN PLANTS

(71) Applicant: NSURE HOLDING B.V., Wageningen (NL)

(72) Inventors: Monique Francisca VAN WORDRAGEN, Arnhem (NL); Peter Albert BALK, Zetten (NL); Apolonia H.M. VAN DER GEEST, Wageningen (NL)

(73) Assignee: NSURE HOLDING B.V., Wageningen (NL)

(21) Appl. No.: 14/789,516

(22) Filed: Jul. 1, 2015

Related U.S. Application Data
(63) Continuation of application No. 11/815,836, filed on Aug. 8, 2007, now abandoned.

(30) Foreign Application Priority Data
Feb. 9, 2005 (EP) 05075317.7

(51) Int. Cl. C12Q 1/68; C07K 14/415; C12N 15/82

(52) U.S. Cl. CPC C12Q 1/6895; C12N 15/8271; C12N 15/8273; C07K 14/415; C12Q 2600/13; C12Q 2600/158

(57) ABSTRACT

The current invention provides a method for rapid testing of stress tolerance in a plant and a method of producing plants with enhanced stress tolerance, in particular cold and/or drought tolerance. Such a method may be applied in breeding and selection programs for this trait, or for determining the timing of induction of stress and cold tolerance or hardening of a plant, for instance for agricultural purposes. The current invention exploits a difference in the temperature and light regimen induced transcriptional regulation of several types of dehydrins, in order to determine a ratio of dehydrin types that is indicative of cold and/or drought tolerance, induction of hardening and dormancy in a plant.

### CROSS-REFERENCE TO RELATED APPLICATIONS

The present application is a continuation of U.S. application Ser. No. 11/815,836, filed Aug. 8, 2007, which U.S. application Ser. No. 11/815,836 is a national phase application under 35 U.S.C. §371 of International Application No. PCT/NL2006/050021, filed Feb. 7, 2006, which claims priority to and the benefit of European Application No. 05075317.7, filed Feb. 9, 2005, the entire contents of all of which applications are incorporated herein by reference.

### FIELD OF THE INVENTION

The current invention relates to the field of plant biology, breeding and agriculture. The invention also relates to methods of determining gene expression levels, in particular methods in involving quantitative amplification or hybridization to oligonucleotides on solid carriers. The invention provides methods for diagnosing plants for, and producing plants with, enhanced stress tolerance, in particular cold and/or drought tolerance.

### BACKGROUND OF THE INVENTION

In plants, expression of many members of the large group of Late Embryogenesis Abundant (LEA) proteins is correlated with desiccation tolerance, and changes in expression are observed in relation to stresses that have a dehydration component. While originally identified in cotton embryos, LEA proteins appeared to be widespread over the plant kingdom and at least six different groups have been identified (Wise and Tunnacliffe 2004). One of the groups comprises the so-called dehydrins (LEA DII family). Dehydrins typically accumulate in plants during dehydration (Close 1996).

Several classes of dehydrins can be distinguished on the basis of structural features. All dehydrins share a highly conserved Lysine-rich sequence, the K-segment (consensus KIKEKLPG). In many dehydrins K-segments are found in more than one copy and in combination with a Serine stretch, the S-segment (consensus SSSSSSSS, usually between 5 and 10 serine residues, mostly 8). The third feature that can be distinguished in many dehydrins is the Y-segment (consensus DEYGNP). S, K and Y segments occur in many different combinations (Campbell and Close 1997, Close 1997). So far, no clear physiological functions have been assigned to dehydrins, although the number of reports that points to specialised functions for individual dehydrins is increasing. Chaperone-like functions have been suggested (Close 1996, Rinne et al. 1999) as well as cryoprotective properties (Momma et al. 2003). Also ion (calcium) binding properties of ERD14 from Arabidopsis (Alsheikh et al. 2003) and a dehydrin like protein from Apium graveolens (Heyen et al. 2002) have been found. Differences in spatial distribution points to specialised functions (Nylander et al. 2001) as well as reports on associations of dehydrins with lipid vesicles (Koag et al. 2003) and plasmodesmata (Karlson et al. 2003).

Dehydrins have also been cloned from angiosperms (Close 1997) and in most cases, they are members of a multigene family (Choi et al. 1999, Nylander et al. 2001 and Porat et al. 2002). The number of dehydrin genes cloned from gymnosperms is increasing. Several LEA cDNA clones were isolated from seeds of Douglas fir (Jarvis et al. 1996) and a dehydrin cDNA was cloned from needles of white spruce (Richard et al. 2000). Besides these, there are cDNA-libraries from Pinus taeda (Johnson 2001, The MIPS database) and Pinus pinaster (Frigerio 2003, NCBI) that contain several dehydrin coding sequences. From Pinus sylvestris two genomic fragments have been isolated that encode dehydrins (Acc. no. AF359133 and AF359134).

A link between dehydrin expression and frost tolerance is suspected because of the dehydration that plays a role at low temperature exposure. Plants growing in the temperate zones have to survive cold periods and show an annual growth rhythm, dictated by changes in photoperiod and temperature. Decreased daylength is generally assumed to be the trigger for growth cessation (Bigras 1996). As dormancy is always associated with frost tolerance, dehydrins are reported to be related to both phenomena (Rowland and Arora 1997, Artlip et al. 1997). In transgenic aspen (Welling et al. 2002) as well as in birch (Welling et al. 2004) it was shown that both photoperiod/daylength and temperature regulate dehydrin expression.

Plants generally contain several dehydrin sequences, which always comprise at least one K segment and optionally further K, Y or S segments/domains (Campbell and Close, 1997). Although the use of dehydrins in general for determining and conferring cold or drought tolerance in plants is widely acknowledged, little information is available about functional differences between the various types of dehydrins.

The use of dehydrin gene encoded polypeptides for conferring cold tolerance and for determining cold tolerance for selection and breeding purposes has been disclosed in U.S. Pat. No. 6,501,006. In this disclosure a Y₂K type dehydrin sequence cloned from Cowpea is used to confer cold tolerance. The Y₂K dehydrin does not contain a stretch of serines such as SK type dehydrins. Y₂K type dehydrins are not present in all plants. In fact, Y-segment containing dehydrins are absent in many cases (Close 1997), which severely limits the application of Y₂K type dehydrins for conferring cold tolerance and/or determining cold tolerance in such plants.

### SUMMARY OF THE INVENTION

The current invention exploits a marked difference in the temperature and light regimen induced transcriptional regulation of SK- and K-type dehydrins, in order to determine stress tolerance, in particular the cold and/or drought tolerance in a plant. Tolerance is not determined in absolute terms of tolerance levels, but rather in terms of relative tolerance levels in comparison to other plants of the same genus or species, for instance in a breeding and selection program for this trait, or for determining the timing of induction of stress tolerance or hardening of the plant, for instance for agricultural purposes. Furthermore, the invention provides methods for increasing stress tolerance, in particular cold tolerance, in a plant by providing a source of a K-type dehydrin. The functional difference between the two types of dehydrins, the K-type dehydrins and SK-type dehydrins, depends on the presence or absence of a serine stretch or S-segment in a dehydrin polypeptide, which is found to have implications for determining and/or conferring stress tolerance, in particular cold or drought tolerance. Preferably the K type and SK type dehydrins disclosed herein do not contain a Y-segment.

Expression of dehydrins is generally associated with cold tolerance in plants and expression of dehydrins is observed to increase during hardening of plants, induced by cold periods or differences in photo-period and -intensity. The current invention discloses a pattern observed in hardening of plants, in particular in gymnosperms, in which during hardening SK-type dehydrins are first (slowly) induced, at the transcriptional level, followed by a decrease in transcription activity in the cold/winter period, when dormancy is induced. Simultaneously with the decrease of SK-type dehydrins, low molecular weight K-type dehydrin messengers become more abundant during the cold period, conferring protection to the plant against low temperatures and drought. Thus an increase in transcriptional activity of SK-type dehydrin genes during hardening is followed by a sharp increase in transcriptional activity of K-type dehydrin genes during the cold period where cold tolerance and dormancy is maximal. The corresponding dehydrin protein levels follow an analogous pattern. The invention exploits the positive correlation with the level and timing of induction of both SK and K-type dehydrins. The differences in transcriptional activity are used to determine a ratio between the SK-type and K-type dehydrins. This ratio may be determined once, to establish the level of stress tolerance in a plant, for instance after a (cold, dark and/or dry) period of hardening, or in a breeding program to select those plants in a group of plants that are most stress tolerant. The ratio may also be determined multiple times, preferably during fixed intervals, to be able to determine the timing and the level of induction of stress resistance, cold tolerance and dormancy in a plant. Absolute numbers for this ratio cannot be given, as they are of course dependent on the particular plant being tested, the particular dehydrins assayed and the sensitivity of the detection technique used. The difference in ratio between plants with relatively low and relatively high levels (compared to each other) of SK-type and K-type dehydrins is indicative of the relative level of stress resistance in the plant. Similarly, observation of a relative shift in the ratio between SK- and K-type dehydrins in a single plant or variety of plants, shifting in time from an induction and relative abundance in SK-type dehydrins towards an induction and relative abundance of K-type dehydrins during acquiring of stress tolerance by a plant or plant variety is key to the current invention.

### BRIEF DESCRIPTION OF THE DRAWINGS

FIGS. 1A-1C: Deduced amino acid sequences of the isolated dehydrins from Pinus sylvestris.

FIG. 1A: Both dehydrins, Psdhn1 and Psdhn2, are compared with a dehydrin sequence of Picea glauca, Pgdhn1.

FIG. 1B: Comparison of the sequences from the low-molecular-weight dehydrins, Psdhn3-8. Conserved domains (Serine-stretch and K-segment) shared by all known dehydrins, are shown in bold. Dots indicate conserved amino acids in the actual comparison.

FIG. 1C: Consensus sequences for the different K-segments found in deduced amino acid sequences from the indicated pine species. For both P. taeda and P. pinaster Accession numbers are given.

FIG. 2A and FIG. 2B: Characteristics of the physiological status of the one-year-old pine seedlings used for expression profiling. Freeze induced shoot electrolyte leakage data are presented for the provenances A70 (open squares) and Lindås (closed squares) together with the Cold Index (B panel, experienced hours below 5° C.). Standard errors of means are given.

FIG. 3: Expression data of dehydrins in pine apical buds from both provenances, using the cDNA-microarray. Microarray data are 2 log ratios calculated from fluorescence values of sample and reference probes of the indicated cDNA-clones. The right panel shows the RT-PCR data obtained from Lindås apical buds. Relative copy numbers were determined by extrapolation from a standard curve and the percentage of the maximal value is given. Standard deviations of duplicate measurements are indicated.

FIGS. 4A-4C: RT-PCR data of dehydrins in pine apical bud material.

FIG. 4A: C_{T}-values (means of duplicates), using the specific primer sets defined in Table 3, the 18S signal was used as the internal control.

FIG. 4B: Calculated fold change in expression relative to week 41 for A70 and to week 43 for Lindås, normalised using the 18S rRNA data. (ΔΔC_{T}=(C_{T,dehydrin}−C_{T,18S})_{Time x}−(C_{T,dehydrin}−C_{T,18S})_{Time 0}). Data are presented in the following order, Psdhn1 (White), Psdhn2 and Psdhn5, for each timepoint.

FIG. 4C: Hierarchical clustering of the C_{T}-values of the three indicated dehydrins.

### DETAILED DESCRIPTION

In a first embodiment the current invention provides a method for determining stress tolerance in a plant, comprising the step of determining the ratio between an SK type dehydrin and a K type dehydrin.

Stress tolerance in this specification is meant to comprise at least the stress factors induced by various field conditions such as drought, light conditions in terms of length and intensity, or low temperatures, as defined by temperatures under 10, 8, 6, 4, 2, 0, −2, −5° C. and below. Stress tolerance also comprises tolerance to drought and/or osmotic stress, which is known to be highly correlated to cold tolerance because of dessication of plants at low temperatures (Environmental Physiology of Plants. A. H. Fitter and R. K. M. Hay, Academic Press, 1987) and will for a large part determine survival and re-growth potential after a cold or dormancy period. A change in light regimen, which may be the result of a decrease in both artificial or natural day length or light intensity, results in a stress or dormancy response in a plant and subsequent hardening and induction of dormancy in a plant. The method according to the invention, determining the ratio between a K-type dehydrin and an SK-type dehydrin, may therefore be carried out once to determine the stress tolerance level in a plant, for instance after a period of hardening. The method may also be carried out multiple times under different temperature and/or daylight conditions to monitor the hardening response and induction of stress tolerance and/or dormancy in a plant. The time intervals of taking samples and determining the ratio may vary. Preferably at least 2, 3, 4, 5, or more samples are collected with intervals of 1, 2, 3, 4, 6 to 8 weeks. During hardening and induction of dormancy in a plant by exposure to a lowering of the temperature and/or a decrease in daylength or (cumulative) light intensity, the ratio between SK/K-type dehydrins will rise slowly (gradual induction of SK-type dehydrins), followed by a marked decrease in the ratio (induction of K-type dehydrins). When determining the stress tolerance of a group of plants, for instance for breeding purposes, those plants from a group of plants having the preferred ratio (having relatively high K-type dehydrins) may be selected. When determining the induction of stress tolerance in a plant by hardening during a season, the induction of hardening (decreased SK/K type ratio, relatively high K-type expression) may be monitored, for instance for agricultural and/or forestry purposes.

The method according to the invention is preferably carried out by detection of an SK₄ type dehydrin and/or even more preferably an SK₂ type dehydrin. However, also other S-domain containing dehydrins with a varying number of K-segments may be suitably applied according to the invention. The K-type dehydrin may be any dehydrin not containing an S-domain and preferably not containing a Y-domain. The K-type dehydrin may contain one or more K-domains and preferably is a K₂ dehydrin, containing 2 K-domains. A K-type domain dehydrin does not comprise S- or Y-domains.

In a preferred embodiment, the method according to the invention is carried out detecting an SK₂ type dehydrin messenger RNA or cDNA molecule having at least 85, 90, 95, 98 or 99% identity with a Psdhn2 (SEQ ID No. 1), or a fragment, homolog or analog thereof, capable of hybridizing under stringent conditions.

In the same or another preferred embodiment the method according to the invention is carried out detecting a K₂ type dehydrin messenger RNA or cDNA has at least 85, 90, 95, 98 or 99% identity Psdhn5 (SEQ ID No. 2), or a fragment, homolog or analog thereof, capable of hybridizing under stringent conditions.

Most preferably, the method according to the invention is carried out on a plant, wherein the plant to be tested for SK-type and K-type dehydrin ratio by mRNA expression or protein content is a gymnosperm, more preferably a gymnosperm of the family of Pinaceae. The invention is particularly suitable for testing of stress tolerance, in particular induction of hardiness, dormancy and/or cold tolerance for plants from the family Pinaceae, in particular from the genus Pinus, the genus Picea, the genus Pseudotsuga, the genus Tsuga, the genus Larix, the genus Abies and the genus Cedrus. Suitable species from these genuses comprise at least: from the genus Pinus in particular: P. halepensis, P. nigra, P. pinaster, P. pinea, P. sylvestris, P. banksiana, P. contorta, P. elliottii, P. flexilis, P. glabra, P. jeffreyi, P. lambertiana, P. ponderosa (syn. P. washoensis), P. radiata, P. taeda, P. lawsonii, P. occidentalis, P. patula), the genus Picea: Picea abies, Picea glauca, Picea engelmannii, Picea sitchensis, Picea pungens)), the genus Pseudotsuga: (Pseudotsuga menziesii (all subspecies), Pseudotsuga lindleyana, Pseudotsuga macrocarpa, Pseudotsuga japonica, Pseudotsuga sinensis (all subspecies)), the genus Larix: (Larix decidua, Larix laricina, Larix occidentalis) the genus Abies: (Abies alba, Abies nordmanniana, Abies procera, Abies fraseri, Abies balsamea), the genus Cedrus: (Cedrus deodara, Cedrus libani) and the genus Tsuga: (T. canadensis, T. caroliniana, T. chinensis, T. diversifolia, T. dumosa, T. forrestii, T. heterophylla, T. mertensiana, T. sieboldii).

The samples obtained from a plant for determining its content of SK-type and K-type dehydrin proteins and dehydrin messenger RNA's may be any part of the plant, such as leaves, flowers, roots, shoots, twigs, fruits, pollen, seeds, embryo's, seedlings, or tissue culture material such as single cells or callus. Particularly preferred is the use of buds, more in particular apical buds.

In a highly preferred embodiment the method according to the invention, the ratio of SK-type and K-type dehydrins is determined by quantitative PCR techniques on mRNA samples which have been converted to cDNA by a reverse transcriptase reaction (RT-PCR). Quantitative PCR may be carried out by conventional techniques and equipment, well known to the skilled person, described for instance in S. A. Bustin (Ed.), et al., A-Z of Quantitative PCR, IUL Biotechnology series, no 5, 2005. Preferably, labeled primers or oligonucleotides are used to quantify the amount of reaction product. Other techniques capable of quantifying relative and absolute amounts of mRNA in a sample, such as NASBA, may also be suitably applied.

A convenient system for quantification is the immunolabeling of the primers, followed by an immuno-lateral flow system (NALFIA) on a pre-made strip (references: Kozwich et al., 2000, Koets et al., 2003 and van Amerongen et al., 2005), for the detection of the SK- and K-type amplification products. As an example, primers specifically capable of selectively amplying the SK-type dehydrin Psdhn2 and the K-type dehydrin Psdhn5 are given in table 3 in example 1. As a positive control for the RNA isolation, reverse transcriptase reaction, amplification reaction and detection step, amplification and detection of a constitutively expressed housekeeping gene may be included in the assay, such as ribosomal (18S) messenger RNA's, actin or GAPDH. Primers may be labeled with direct labels such as FITC (fluorescein), Texas Red, Rhodamine and others or with tags such as biotin, lexA or digoxigenin which may be visualized by a secondary reaction with a labeled streptavidin molecule (for instance with carbon or a fluorescent label) or a labeled antibody (labeled with fluorescent molecules, enzymes, carbon, heavy metals, radioactive isotopes or with any other label).

In another embodiment, comparative hybridization is performed on mRNA or cDNA populations obtained from a plant or sample thereof, to one or more dehydrin type-specific sequences, which may optionally be tagged or labeled for detection purposes, or may be attached to a solid carrier such as a DNA array or microarray. Suitable methods for microarray detection and quantification are well described in the art and may for instance be found in: Applications of DNA Microarrays in Biology. R. B. Stoughton (2005) Annu. Rev. Biochem. 74:53-82, or in David Bowtell and Joseph Sambrook, DNA Microarrays: A Molecular Cloning Manual, Cold Spring Harbor laboratory press, 2003.

The invention also pertains to nucleic acid carriers, such as arrays and microarrays or DNA chips, comprising nucleotides on a glass, plastics, nitrocellulose or nylon sheets, silicon or any other solid surface, which are well known in the art and for instance described in Bowtell and Sambrook, 2004 (ibid) and in Ausubel et al., Current protocols in Molecular Biology, Wiley Interscience, 2004. A carrier according to the current invention comprises at least two (oligo-) nucleotide probes capable of selectively hybridizing with SK-type and K-type dehydrins. Preferably, the SK hybridizing (oligo-)nucleotide probe is or is a fragment derived of SEQ ID No. 1 and/or the K type hybridizing (oligo-) nucleotide probe is or is a fragment derived of SEQ ID No. 2.

In another embodiment, the method according to the invention and in particular the carrier comprising DNA fragments capable of selectively hybridizing to SK or K type dehydrins, may further comprise additional sequences that are known to be involved in stress or cold tolerance in plants. The current inventors have found the expression of newly identified sequences, SEQ ID No's 3 and 4, to be indicative of induction and/or increased levels of stress tolerance in plants. These sequences according to this invention have been found to be indicative for induction of cold tolerance and/or hardening and capable of increasing hardiness and cold tolerance, in particular in Pinaceae. The methods and the DNA carrier according to the invention may therefore further comprise an oligonucleotide sequence capable of selectively amplifying and/or hybridizing with sequences having at least 85, 90, 95, 98, 99% identity with a sequence encoding Pinus cold tolerance related transcript (SEQ ID No. 3). This sequence is induced in stress or cold tolerant plants. The carrier may also further comprise an oligonucleotide sequence capable of hybridizing with sequences having at least 85, 90, 95, 98, 99% identity with a sequence encoding a Pinus alpha tubulin transcript (SEQ ID No. 4). The relative abundance of this sequence is decreased in cold or stress tolerant plants.

In another embodiment, the method according to the invention is carried out on samples obtained from a plant, whereby the ratio of SK-type and K-type dehydrins is determined by comparative analysis of protein samples obtained from the plant, for instance using mass spectrometry techniques, or preferably using dehydrin type specific antibodies. Such antibodies may optionally be labeled, tagged and/or attached to a (solid) carrier. Type specific antibodies for K-type and SK-type dehydrins are available in the art, Close, T. J., Fenton, R. D. and Moonan, F. (1993) Plant Mol. Biol. 23:279-286 and available from, for instance; (Stressgen Biotechnologies Corp., Victoria, Canada). Suitable antibodies may also be generated by the skilled artisan. Generation of monoclonal or polyclonal antibodies capable of interacting with a specific protein or a (unique) fragment or sequence in that protein is a standard technique which can be found in many textbooks, such as Current Protocols in Immunology, Wiley Interscience, 2004.

The method according to the invention will be most usefull for a rapid stage determination of stress tolerance in nursery- and plantbreeding practice, in particular for Pinaceae and for forestry purposes. Determining the level of stress tolerance will be most suitable for testing Pinaceae seedlings in the autumn, before they are taken from the field and stored till spring in a climatised winter storage at approximately −2° C. to +4° C. The level of hardening and hence the optimal timing for storage is determined by the method according to the invention. The ratios will be usefull as indicators for different stages of hardening or dormancy and the resulting stress tolerance, in particular frost tolerance and winter hardiness and re-growth potential in spring, for instance after winter storage.

The current invention also provides methods and means for enhancing stress tolerance in a plant, preferably a gymnosperm plant and most preferably of the genus Pinaceae, the method comprising the step of providing to the plant a source of K-type dehydrins, in particular K₂ dehydrins, in order to decrease the ratio between SK-type and K-type dehydrins. Preferably the source is a gene encoding K-type dehydrins, the gene being operably linked with regulatory sequences capable of conferring expression and translation in the host plant. The gene may be comprised in an expression cassette in a vector, preferably an Agrobacterium vector or a viral vector known in the art of producing transgenic plants. Methods for transformation of Pinaceae using vectors or particle bombardment are for instance described in Tian et al., Planta, 213(6) 2001, Tang and Tian, J. Exp. Botany, vol 54 no. 383, 2003, Aronen T S et al., Transgenic Res. 12(3), 2003 and in Grant J E et al., Plant Cell reports, vol. 22 no. 12, 2004.

The promoter driving the expression of the K-type dehydrin may be a constitutive promoter, for instance a viral promoter (such as CaMV) or an inducible or regulatable promoter. Most preferably, the promoter is a temperature sensitive or temperature inducible promoter. Temperature dependent regulation of gene expression in plants is well described in the field and the selection and application of a temperature dependent promoter for a given plant of interest is well within the ability of a skilled artisan.

The invention thus provides a method to obtain transgenic plants, in particular transgenic Pinaceae, with an increased stress/cold tolerance and/or an accelerated and enhanced hardening, comprising a recombinant expression cassette comprising a promoter operably linked to a gene providing expression of a K-type dehydrin, preferably a gene having at least 85, 90, 95, 98, 99% identity with a gene encoding a K2-type dehydrin from Pinus sylvestris, SEQ ID No. 2 (Psdhn5), SEQ ID No. 5 (Psdhn 3), SEQ ID No. 6 (Psdhn 4), SEQ ID No. 7 (Psdhn 6), SEQ ID No. 8 (Psdhn 7) and SEQ ID No. 9 (Psdhn 8).

In another aspect the present invention relates to a “kit” containing elements for use in the methods of the invention. Such a kit may comprise a carrier to receive therein one or more containers, such as tubes or vials. The kit may further comprise unlabeled or labeled oligonucleotide sequences of the invention, e.g. to be used as primers, probes, which may be contained in one or more of the containers, or present on a carrier. The oligonucleotides may be present in lyophilized form, or in an appropriate buffer. One or more enzymes or reagents for use in isolation of nucleic acids, purification, restriction, ligation and/or amplification reactions may be contained in one or more of the containers. The enzymes or reagents may be present alone or in admixture, and in lyophilised form or in appropriate buffers. The kit may also contain any other component necessary for carrying out the present invention, such as manuals, buffers, enzymes (such as preferably reverse transcriptase and a thermostable polymerase), pipettes, plates, nucleic acids (preferably labeled probes), nucleoside triphosphates, filter paper, gel materials, transfer materials, electrophoresis materials and visualization materials (preferably dyes, labeled antibodies or -enzymes) autoradiography supplies. Such other components for the kits of the invention are known per se.

### DEFINITIONS

### Hardening and Tolerance to Stress Factors

In order to tolerate the stresses that they face, plants have to adapt their metabolism. Several cellular and metabolic functions are altered by low temperatures and freezing. Plant membranes undergo both qualitative and quantitative modifications during periods of cold acclimation and deacclimation. The lipid composition of the plasma membrane and chloroplast envelopes changes during cold acclimation. Carbohydrate content is known to vary according to the hardening status of a tissue. Starch concentrations decrease and the concentrations of soluble sugars increase in cold acclimating tissues of woody plants. The process of adaptation to low temperatures causes changes in the function of genes and proteins. The adaptation involves the modification of pre-existing proteins and the up- and down-regulation of gene expression or protein synthesis. Cold induced gene activity aids in the metabolic adjustment to low temperatures or confer freezing tolerance to tissues. In woody plants, many of the genes and proteins related to cold acclimation may also be connected to the dormancy status of the plants. Late Embryogenesis Abundant (LEA) proteins, in particular the D-11 family or group 2 LEA proteins, are called dehydrins. Dehydrin proteins are induced in plants by dehydration-related environmental stresses such as low temperature, drought or high salinity. These proteins, of variable molecular masses, have been found in many plant species. Indicative of dehydrins is the presence of one or several lysine-rich units called the K-segments conserved near the carboxy terminus of the protein and repeated several times throughout the sequence. Some dehydrins also possess a string of serine residues (S-segment). Another consensus sequence (DEYGNP), the Y-segment, can be found near the amino terminus of some of the dehydrins.

A DNA segment, in particular a segment containing a dehydrin encoding gene, is “operably linked” when it is placed into a functional relationship with another DNA segment. For example, a promoter or enhancer is operably linked to a coding sequence if it stimulates the transcription of the sequence. DNA for a signal sequence is operably linked to DNA encoding a polypeptide if it is expressed as a preprotein that participates in the secretion of the polypeptide. Generally, DNA sequences that are operably linked are contiguous, and, in the case of a signal sequence, both contiguous and in reading phase. However, enhancers need not be contiguous with the coding sequences whose transcription they control. Linking is accomplished by ligation at convenient restriction sites or at adapters or linkers inserted in lieu thereof.

“Sequence identity” is herein defined as a relationship between two or more amino acid (polypeptide or protein) sequences or two or more nucleic acid (polynucleotide) sequences, as determined by comparing the sequences. In the art, “identity” also means the degree of sequence relatedness between amino acid or nucleic acid sequences, as the case may be, as determined by the match between strings of such sequences. “Similarity” between two amino acid sequences is determined by comparing the amino acid sequence and its conserved amino acid substitutes of one polypeptide to the sequence of a second polypeptide. “Identity” and “similarity” can be readily calculated by known methods, including but not limited to those described in (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heine, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48:1073 (1988).

Preferred methods to determine identity are designed to give the largest match between the sequences tested. Methods to determine identity and similarity are codified in publicly available computer programs. Preferred computer program methods to determine identity and similarity between two sequences include e.g. the GCG program package (Devereux, J., et al., Nucleic Acids Research 12 (1): 387 (1984)), BestFit, BLASTP, BLASTN, and FASTA (Altschul, S. F. et al., J. Mol. Biol. 215:403-410 (1990). The BLAST X program is publicly available from NCBI and other sources (BLAST Manual, Altschul, S., et al., NCBI NLM NIH Bethesda, Md. 20894; Altschul, S., et al., J. Mol. Biol. 215:403-410 (1990). The well-known Smith Waterman algorithm may also be used to determine identity.

Preferred parameters for polypeptide sequence comparison include the following: Algorithm: Needleman and Wunsch, J. Mol. Biol. 48:443-453 (1970); Comparison matrix: BLOSSUM62 from Hentikoff and Hentikoff, Proc. Natl. Acad. Sci. USA. 89:10915-10919 (1992); Gap Penalty: 12; and Gap Length Penalty: 4. A program useful with these parameters is publicly available as the “Ogap” program from Genetics Computer Group, located in Madison, Wis. The aforementioned parameters are the default parameters for amino acid comparisons (along with no penalty for end gaps).

Preferred parameters for nucleic acid comparison include the following: Algorithm: Needleman and Wunsch, J. Mol. Biol. 48:443-453 (1970); Comparison matrix: matches=+10, mismatch=0; Gap Penalty: 50; Gap Length Penalty: 3. Available as the Gap program from Genetics Computer Group, located in Madison, Wis. Given above are the default parameters for nucleic acid comparisons.

Optionally, in determining the degree of amino acid similarity, the skilled person may also take into account so-called “conservative” amino acid substitutions, as will be clear to the skilled person. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; a group of amino acids having acidic side chains is aspartic acid and glutamic acid and a group of amino acids having sulphur-containing side chains is cysteine and methionine. Preferred conservative amino acids substitution groups are: valine-leucine-isoleucine, phenylalanine-tyrosine, lysine-arginine, alanine-valine, and asparagine-glutamine. Substitutional variants of the amino acid sequence disclosed herein are those in which at least one residue in the disclosed sequences has been removed and a different residue inserted in its place. Preferably, the amino acid change is conservative. Preferred conservative substitutions for each of the naturally occurring amino acids are as follows: Ala to ser; Arg to lys; Asn to gln or his; Asp to glu; Cys to ser or ala; Gln to asn; Glu to asp; Gly to pro; His to asn or gln; Ile to leu or val; Leu to ile or val; Lys to arg; gln or glu; Met to leu or ile; Phe to met, leu or tyr; Ser to thr; Thr to ser; Trp to tyr; Tyr to trp or phe; and, Val to ile or leu.

Nucleic acid sequences encoding dehydrin polypeptides and/or stress tolerance conferring activity may also be defined by their capability to hybridise with the (complementary strand of) the nucleotide sequence of SEQ ID No's: 1, 2, 5, 6, 7, 8, 9 or 10, preferably under moderate, or more preferably under stringent hybridisation conditions. Stringent hybridisation conditions are herein defined as conditions that allow a nucleic acid sequence of at least about 25, preferably about 50 nucleotides, 75 or 100 and most preferably of about 200 or more nucleotides, to hybridise at a temperature of about 65° C. in a solution comprising about 1 M salt, preferably 6×SSC or any other solution having a comparable ionic strength, and washing at 65° C. in a solution comprising about 0.1 M salt, or less, preferably 0.2×SSC or any other solution having a comparable ionic strength. Preferably, the hybridisation is performed overnight, i.e. at least for 10 hours and preferably washing is performed for at least one hour with at least two changes of the washing solution. These conditions will usually allow the specific hybridisation of sequences having about 90% or more sequence identity. Moderate conditions are herein defined as conditions that allow a nucleic acid sequences of at least 50 nucleotides, preferably of about 200 or more nucleotides, to hybridise at a temperature of about 45° C. in a solution comprising about 1 M salt, preferably 6×SSC or any other solution having a comparable ionic strength, and washing at room temperature in a solution comprising about 1 M salt, preferably 6×SSC or any other solution having a comparable ionic strength. Preferably, the hybridisation is performed overnight, i.e. at least for 10 hours, and preferably washing is performed for at least one hour with at least two changes of the washing solution. These conditions will usually allow the specific hybridisation of sequences having up to 50% sequence identity. The person skilled in the art will be able to modify these hybridisation conditions in order to specifically identify sequences varying in identity between 50% and 99%.

### EXAMPLES

### Materials and Methods

### Plant Material for Cloning Purposes

Apical bud material used for the construction of the cDNA Expression library was collected in week 48 of 1997 from two-year-old containerised Pinus sylvestris, grown outdoors in the Netherlands. The plants were obtained from Applied Plant Research (Boskoop, The Netherlands).

For construction of the subtraction libraries, apical buds from two-year-old Pinus sylvestris plants of the Dutch provenance Vosterbos were used. The containerised plants were obtained via a local grower and were grown outside. February the fifth (2001) apical buds were collected from 30 plants and frozen in liquid nitrogen (quiescent sample). At the same time another 30 plants were subjected to cold treatment during the night. Plants were put in a dark climate-controlled room where temperature decreased gradually from 20° C. to −10° C. The plants were kept at −10° C. for five hours. Relative humidity varied between 80 and 90%. The next morning apical buds were collected from these plants and frozen in liquid nitrogen (cold treated sample). From the same batch of plants, thirty apical buds were collected and frozen in liquid nitrogen at the third of April 2001, shortly before bud-break (release sample).

### Plant Material for Expression Analysis

For expression analysis two provenances were used. A commercial British seedlot, A70 (55°28′N to 57°41′N) and seed from a Norwegian provenance, Lindås (60°42′N; 5°19′E). Both were sown on 14 May 2001 in HIKO 120-ss containers filled with peat (Pindstrup 2) and raised in a greenhouse in Aarslev, Denmark. During the greenhouse period the seedlings were irrigated regularly and liquid fertiliser (Pioner NPK Makro: 100 ppm N, 25 ppm P, 75 ppm P, 75 ppm K, 30 ppm Mg) was applied three times per week. On 10 August the seedlings were placed outside where they remained for the rest of the experimental period. After this date they were irrigated regularly but not fertilised. Every two weeks, starting in week 37 (10 September) and until week 4 (21 January), apical buds of 50 randomly selected seedlings were harvested and frozen in liquid nitrogen. At the same time shoot tip samples were tested for frost tolerance using freeze induced electrolyte leakage.

The Cold Index was determined by taking the air temperature (Tₐᵢᵣ) during the period from week 37 to week 4, as measured 0.5 m above the ground every half hour using a datalogger (Tinytalk, Gemini Dataloggers, Chichester, UK). A cold index (Ci)) accumulating the number of hours with Tₐᵢᵣ<5° C. was calculated from the temperature data.

### Freeze Testing

Shoot tip samples were tested for frost tolerance. On each date 30 randomly selected seedlings were sampled, and a 2-cm long stem segment (with needles) was excised from each seedling immediately below the apical bud. The segments were washed in tap water and subsequently rinsed in deionised water. Each sample was then placed in a 20-ml plastic bottle and capped. Half of the samples (15 replicates) were frozen in a programmable freezer at a rate of 2° C. h-1 to −15° C. They were held at this temperature for 120 minutes before they were removed from the freezer and placed at −1° C. overnight. The samples were taken out the following morning for assessment of frost injuries. The remaining 15 samples were held at +2° C. as unfrozen control until freeze treatments had ended.

For the evaluation of frost injury, shoot electrolyte leakage of the frost treated samples and unfrozen controls was assessed using the conductivity method modified from McKay (1992). After rewarming the samples to room temperature, 20 ml of deionised water with low electrical conductivity (CO) was added to each bottle. The samples were subsequently incubated in darkness at room temperature for 24 hours±15 min. After inverting the incubated samples five times, the conductivity of the water (C1) was measured with a conductivity meter. The samples were then autoclaved at 120° C. and 1.2 bar for 60 minutes to kill the cells. After cooling the samples to room temperature, a second recording of conductivity (C2) was made. The shoot electrolyte leakage (SEL) was calculated as relative conductivity (RC):

$$RC = \frac{{C}_{1} - {C}_{0}}{{C}_{2} - {C}_{0}} \cdot 100 \%$$

The electrolyte leakage caused by freezing was expressed as the difference between RC of a frozen sample and RC of a corresponding control sample and designated SELdiff-15. A decline in SELdiff with time would then indicate that the shoot tissue had become more frost tolerant to −15° C. (Lindström & Håkansson 1996, Stattin et al. 2000).
cDNA Library Construction

RNA was isolated from frozen buds according to Chang et al. (1993). mRNA was extracted using the Dynabeads mRNA Purification Kit (Dynal Biotech, Germany) and used for the construction of the cDNA libraries. The cDNA Expression library (EL) was made using the λ ZAP II system (Stratagene, The Netherlands) following manufacturers instructions.

Subtraction libraries were made using the PCR Select cDNA Subtraction Kit (BD Biosciences Clontech, Europe) according to manufacturers' instructions. Two different libraries were made in order to enrich for genes that were either specifically expressed during quiescence or as a result of the applied short-term low temperature treatment. In order to enrich for quiescence related genes, RNA from the quiescent sample was used as tester whereas a mixture of equal portions of RNA from the cold treated and the release samples was used as driver resulting in quiescent enriched library (QL). For the enrichment of the cold induced genes, RNA from the cold treated sample was used as tester whereas a mixture of equal portions of RNA from the quiescent and the release sample was used as driver resulting in cold enriched library (CL). The Advantage PCR Kit (BD Biosciences Clontech) was used for the amplification of the libraries. PCR conditions were according to the manufacturer protocol. The products, ranging in size from three hundred up to thousand basepairs, were cloned in the pGEM-T Easy Vector (Promega, The Netherlands).

Isolation of Dehydrin cDNA Clones

From the Expression library 1·10⁵ plaques were screened using the picoBlue Immunoscreening Kit (Stratagene, The Netherlands) and the Anti-Dehydrin polyclonal antibody from Stressgen (Canada). Positive plaques were isolated and the vector (pBluescript SK⁻) containing the putative dehydrin cDNA was excised following the protocol of Stratagene. The clones were sequenced and identified using BLAST available on the web.

Besides, randomly selected clones from the Expression library (1000 clones) and both the Subtraction libraries (500 clones each) were sequenced and identified using BLAST. Contig analysis of dehydrin related clone sequences was done using DNASIS Ver. 2.6 (Hitachi Software Engineering Co.).

In order to isolate missing parts at the 3′- or 5′-ends of the clones, RACE reactions were performed. The GeneRacer Kit, purchased from Invitrogen (Life Technologies, The Netherlands), was used. Pooled RNA from the whole range of samples taken from the one-year-old Lindås seedlings was used to make cDNA following manufacturers' instructions.

### Expression Analysis Using the Microarray

A microarray was prepared using 1500 clones selected from all three cDNA libraries. A selection of the identified dehydrins was spotted on the microarray. To monitor gene expression during autumn and winter, a Cy5-labeled cDNA population was prepared from each of the samples taken from the two mentioned provenances. For each hybridisation, a single Cy3-labeled cDNA population, prepared from unrelated Scots pine bud material collected in the Netherlands, was used as a common reference. Hybridisation procedures were as described by Aharoni et al. (2000) and van Doom et al. (2004). Relative expression of each individual clone is presented as the 2 log ratio calculated from the fluorescence value of sample and reference probes.

### RT-PCR Measurements

In order to verify the data obtained from the microarray, Realtime RT-PCR was used. Total RNA was isolated as described before and RNA preparations were treated with DNAseI (AP Biotech) and purified using the RNeasy system (Qiagen, Westburg, The Netherlands). Half a microgram of pure total RNA was used for the synthesis of cDNA. Anchored oligo (dT)₂₃ from SIGMA (The Netherlands) was used in combination with Superscript II Reverse Transcriptase from Invitrogen (Life Technologies, The Netherlands). Dilutions of this cDNA were used for Realtime PCR using the qPCR Core Kit (Eurogentec, Belgium) and the iCycler system (BIORAD Laboratories, The Netherlands). Primer sets used are described in Table 3. The annealing temperature was chosen in such a way that no by-products were being produced, as judged from melt-curve analysis and agarose gel-electrophoresis. PCR efficiencies were between 95 and 110% measured by using serial dilutions of pure target. PCR product formation was detected using Sybr Green I intercalation. A threshold was set, which intersected the amplification curves in the linear region of the semi-log plot. The point at which the curve crosses the threshold, the so-called threshold value (C_{T}), was taken as a measure for the relative amount of target present in a certain sample. Relative changes in gene expression were calculated using the 2^{-ΔΔC}_{T} method described by Livak and Schmittgen (2001). The signal obtained from a PCR on the same batch of pine cDNA using primers homologous to Arabidopsis 18S rRNA, was taken as the reference for normalisation. The sequences of the 18S rRNA primers were as follows, 5′TGACGGAGAATTAGGGTTCG and 5′CCTCCAATGGATCCTCGTTA. The expected size of the PCR fragment was 195 base pairs, based on sequence information from the Arabidopsis sequence. Hierarchical clustering of realtime RT-PCR data was performed using Acuity 3.1 software (Axon Instruments Inc.).

### Results

### Example 1

### Introduction

Pinus sylvestris (Scots pine) seedlings display a strictly periodic growth pattern over the year. When seedlings are placed under different light regimes, growth cessation and bud-set always takes place (Christersson 1978, Ekberg et al. 1979). Growth cessation and frost tolerance develop in parallel (Dormling 1993) and are triggered by both short days and low temperature, especially in one-year-old seedlings (Christersson 1978, Repo et al. 2000).

For seedlings of Pinus sylvestris different stages of hardening have been reported. As growth ceases the bud enters a stage of ecodormancy or quiescence (Dormling 1993, Lang 1987). This stage is characterised by intermediate frost tolerance levels. The timing of this first stage varies among pines originating from different latitudes. Growth ceases earlier in northern provenances compared to southern provenances (Repo et al. 2000). This seems to be directly influenced by differences in photoperiod (Ekberg 1979). As hardening proceeds, a second stage is entered in which buds develop rest (endodormancy, Lang 1987) and frost tolerance is at its maximal level (Dormling 1993). Variation in rates of hardening was found between provenances from different latitudes (Repo et al. 2000). The final level of frost tolerance seems to be influenced primarily by the actual cold sum and not so much by latitudinal origin.

Seasonal variation in dehydrin protein content has been studied in different tissues of Scots pine (Pinus sylvestris) and several different dehydrins have been identified using a K-segment specific antibody (Close et al. 1993, Kontunen-Soppela and Laine 2001). It was found that dehydrin proteins are present throughout the year but levels vary in a tissue specific manner. In bud tissue a 60 kDa dehydrin is present at high levels during the winter.

This specification provides the cDNA-cloning of several dehydrins, representing different classes, from dormant apical buds of Scots pine. The expression profile of a selection of these genes was studied during autumn and winter in two provenances from different latitudes. The results indicate distinct roles for the different dehydrin types K and SK in the protection of pine buds during this period. The changes in gene expression, SK-/K-type ratio and the differences in timing of those changes between the two provenances in relation to frost tolerance are exploited to determine cold tolerance and conferring cold tolerance to a plant.

Identification of Dehydrin cDNA Clones

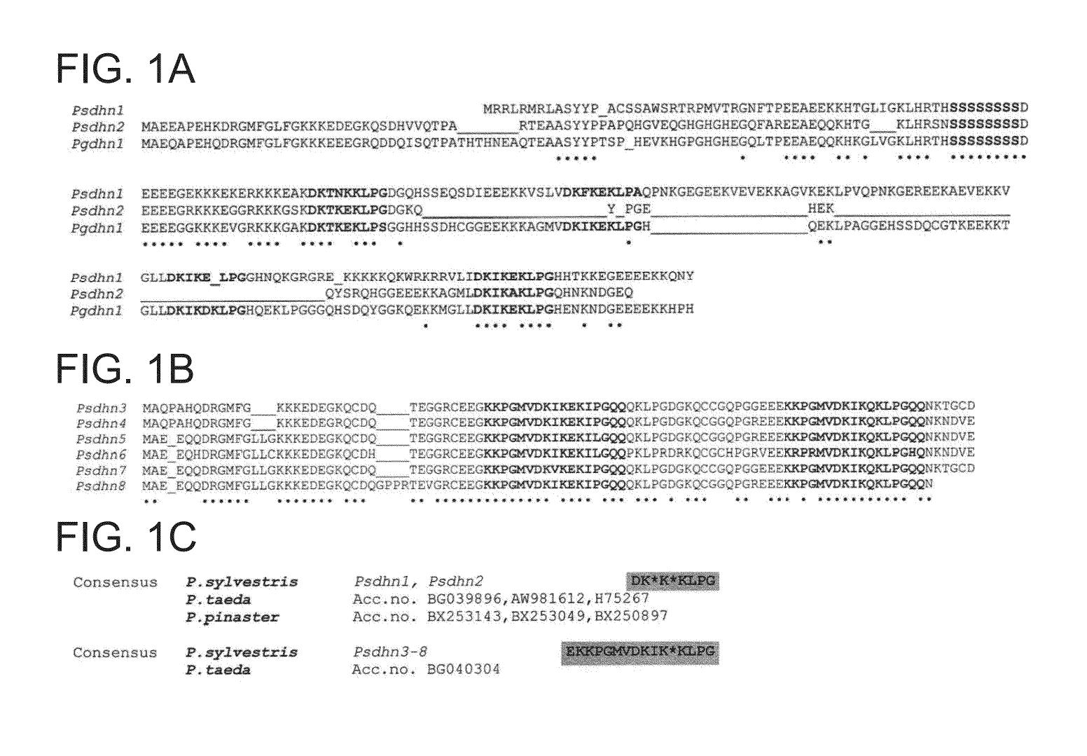

Immunoscreening of the EL cDNA library resulted in the isolation of four clones that show high homology with known dehydrin sequences present in the public databases. Based on sequence similarity with a dehydrin from white spruce, Pgdhn1 (Acc. no. AF1009916), one of the clones, designated Psdhn1, was probably full-length. Since the deduced amino acid sequence contained an eight-serine stretch and four conserved repeats of the lysine-rich K-segment (Table 1 and FIG. 1A, SEQ ID No. 10), it could be classified as a SK₄ type dehydrin. The amino acid sequence displayed homology with sequences derived from the genomic fragments isolated by Mikkonen (Acc. no. AF359133 and AF359134). Both deduced amino acid sequences contain an S-segment and four K-segments, like Psdhn1. Predictions with regard to (post translational) modifications were made using the PredictProtein server (http://cubic.bioc.columbia.edu/predictprotein/predictprotein.html). Results are presented in Table 2. In the case of Psdhn1 several possible locations for phosphorylation were found and one putative myristoylation site. Additionally, a nuclear localisation signal could be recognised.

TABLE 1
Characteristics of the dehydrin cDNA-clones isolated from the libraries.

| | total number | number of cDNA clones per library | | | | | | | | |
|---|---|---|---|---|---|---|---|---|---|---|
| cDNA- | of cDNA | expression | enriched | enriched | | | | conserved domains | | |
| clone | clones | library | quiescent | cold | full-length | MW (kD) | pI | S | K | Acc. no. |
| Psdhn1 | 1 | 1 | | | yes | 26 | 10.2 | 1 | 4 | AJ289610 |
| Psdhn2 | 1 | 1 | | | yes | 14 | 6.9 | 1 | 2 | AJ512361 |
| Psdhn3 | 8 | 6 | 2 | | yes | 10 | 8.4 | — | 2 | AJ512362 |
| Psdhn4 | 2 | | 2 | | yes | 10 | 8.9 | — | 2 | AJ512363 |
| Psdhn5 | 20 | 11 | 7 | 2 | yes | 10 | 7.9 | — | 2 | AJ512364 |
| Psdhn6 | 1 | 1 | | | yes | 11 | 9.0 | — | 2 | AJ512365 |
| Psdhn7 | 8 | 7 | 1 | | yes | 10 | 7.7 | — | 2 | AJ512366 |
| Psdhn8 | 1 | | 1 | | no | | | — | 2 | AJ512367 |

Besides the total number of clones that represent each dehydrin, the cDNA-library from which each individual originates, is indicated. Values for the Molecular Weights (MW) and the Isoelectric Point (pI) are calculated from deduced amino acid sequences.

TABLE 2
Motifs found in the protein sequences of a selection of the cloned pine dehydrins.

| dehydrin | type | amidation | myristoylation | glycosylation | phosphorylation | other |
|---|---|---|---|---|---|---|
| Psdhn1 | SK₄ | | 91 (1) | | 32, 39, 58, 83, 98, 104, 107, 209 (8) | Nuclear Localization Signal; |
| | | | | | | GREKKKKKQKWRKRR |
| Psdhn2 | SK₂ | 17, 95, 102 (3) | 51 (1) | 82 (1) | 75, 88, 106, 116 (4) | |
| Psdhn7 | K₂ | 14, 35 (2) | 65 (1) | 90 (1) | 92 (1) | Putative disulfide bonding, (position 25) |

Predictions are made using the Predict Protein facility available on the web (Columbia University Bioinformatics Centre). Amino acid positions are given, number of motifs between brackets.

Two cDNA clones showed homology with other dormancy- and desiccation-related proteins. Although the signals were less intense than the signals of the dehydrin homologues, they could be easily recognised. One of the clones was related to DRM1 from Pisum sativum (Acc. no. AF029242) and the other one was similar to clone PCC 13-62 from Craterostigma planagineum (Acc. no. P22242). Both cDNA-sequences encode for lysine rich proteins but they do not contain the K-segment specific consensus sequence.

From the 2000 sequenced clones, randomly selected from the three cDNA-libraries, 5% were identified as dehydrin homologues. Fifty of them were picked from the Expression library, forty-five from the library enriched for quiescent genes and 7 from the library enriched for low temperature induced genes. Contig analysis revealed 7 additional distinct dehydrin genes, designated Psdhn2 through Psdhn8 (Table 1 and FIGS. 1A-1C). High numbers of Psdhn3, Psdhn5 and Psdhn7 were picked (Table 1).

Based on sequence similarities it was concluded that part of the Psdhn2 (SEQ ID No. 1) and Psdhn8 (SEQ ID No. 9) sequence information was missing. However, Psdhn8 appeared to be almost full length whereas Psdhn2 was most probably missing a considerable part of the 5′ end. So therefore we focused on getting additional sequence information only for Psdhn2. The primer that was chosen for this purpose was similar to the one used for RT-PCR (Table 3). The deduced amino acid sequence of the extra fragment was 56 residues long and had high homology with the dehydrin from white spruce, Pgdhn1 (FIG. 1A). Psdhn2 was classified as an SK₂-type dehydrin. The consensus sequence of the K-segments present in both Psdhn1 and Psdhn2 was much alike the sequences found in several dehydrins of different pine species (FIG. 1C). Psdhn2 showed high homology, 72 percent at the nucleotide level, with all published dehydrin sequences from Pinus pinaster.

TABLE 3
Primer sequences used for RT-PCR and RACE reactions.
Primerset Psdhn3-8 detects all mRNA molecules from the corresponding
genes simultaneously.

| | | | appliedTₐₙₙₑₐₗ | Productsize | |
|---|---|---|---|---|---|
| Target | Primersequence | Orientation | (° C.) | (bp) | Position |
| Psdhn1 | 5′CACACGGGTTTGATAGG | Forward | 55 | 171 | within |
| Psdhn1 | 5′TGATCTGAAGAATGCTGTCC | Reverse | | | coding region |
| Psdhn2 | 5′TGAGAATAATGGTACGTGCGT | Forward | 55 | 156 | 3′untranslated |
| | GTTG | | | | region |
| Psdhn2 | 5′CTGAAGGTAAGCTCGTACCGA | Reverse | | | |
| | AACC | | | | |
| Psdhn5 | 5′GTTCAGGGCATTGCTTAGGAG | Forward | 55 | 129 | 3′untranslated |
| | | | | | region |
| Psdhn5 | 5′GCAAATACCGACCTCACCATC | Reverse | | | |
| Psdhn3-8 | 5′GGAAGAAACCGGGAATGG | Forward | 55 | 163 | within |
| Psdhn3-8 | 5′TTTTGTTGTCCCGGCAGC | Reverse | | | coding region |

Psdhn3-8, represented by high numbers of clones mainly present in both the Expression library and the library enriched for quiescent cDNA clones, displayed a high degree of homology (FIG. 1B). They all contained two K-segments so they were classified as K₂-types. Based on 5′RACE reactions, using the group-specific primer (Table 3), these clones were judged to be full-length. A specific EST from Pinus taeda (BG040304) displayed high homology, 75 percent at the nucleotide level, with this group of dehydrins. The 3′K-segment consensus sequence appeared to be more conserved between the species (FIG. 1C) than did the 5′K-segment. The consensus sequence is clearly more extensive than the consensus sequences of the K-segment found in Psdhn1 and Psdhn2.

Predictions with regard to putative protein modifications of Psdhn2-8 were made using the PredictProtein server and the results are shown in Table 2 (Psdhn1 being the representative of the whole group of K₂-type dehydrins). Several kinds of modifications are possible and the only difference between Psdhn2 (SEQ ID No. 1) and Psdhn3-8 is that the latter ones are predicted to form disulfide bonds. The amino acid sequences of PSDHN 1-8 proteins can be found in SEQ ID No's 11 to 18 respectively.

### Example 2

### Characteristics of the Physiological Status of the Pine Seedlings Used for Transcriptional Profiling

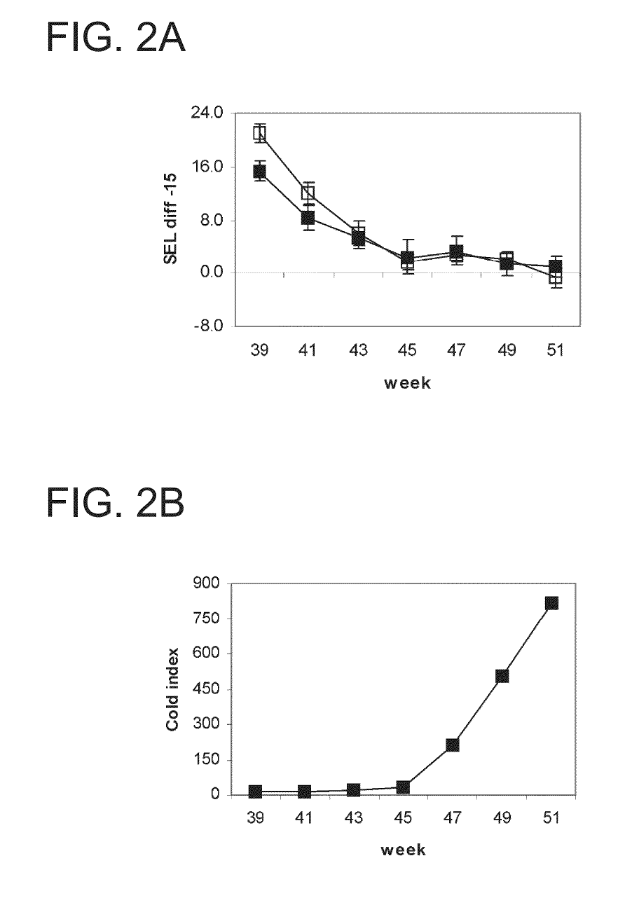

Results of frost tolerance measurement of apical shoots for both pine provenances, A70 and Lindås are shown in FIGS. 2A-2B. At week 39 the SEL diff-15 value was found to be higher in A70 compared to Lindås. From week 43 on, there is no significant difference between the two provenances examined. Both remained frost tolerant for the rest of the experimental period. Comparison of the frost tolerance data with the Cold Index (experienced hours below 5° C.) showed that the shoots have reached their maximal level of frost tolerance prior to a sharp increase in experienced cold.

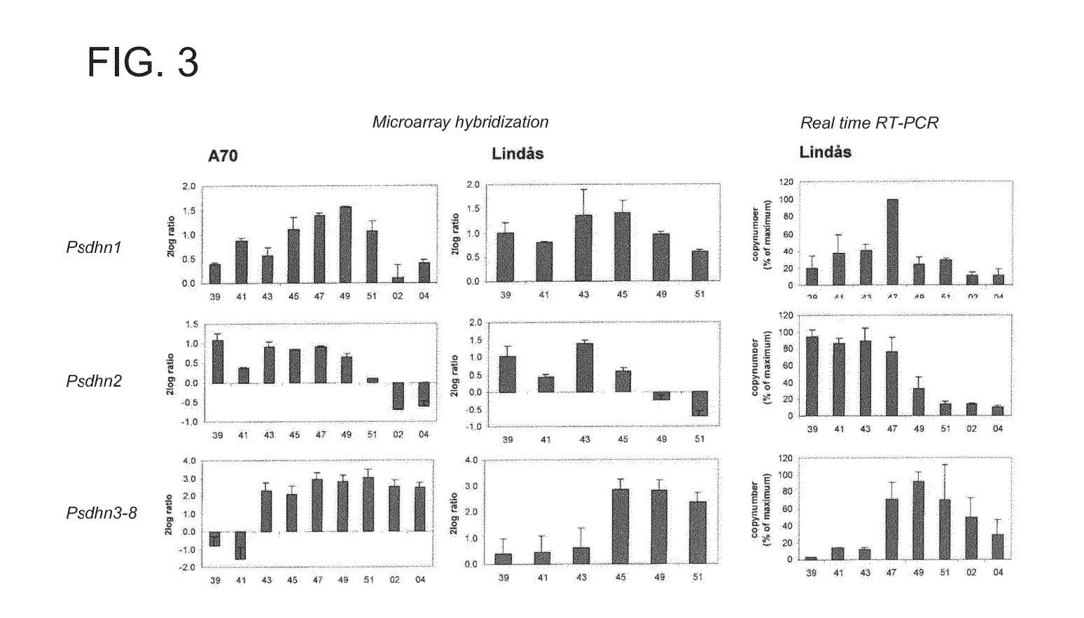

Expression of dehydrins in pine apical buds was monitored from week 39 2001 until week 04 2002. Results from the microarray hybridisations are presented in FIG. 3. Data from Psdhn1 and that of a highly homologous clone, 95% at the nucleotide level, were taken together and the same holds for Psdhn2, in this case the homology between the two clones was 85%, enough to get cross-hybridisation. Eleven clones on the microarray represent dehydrins Psdhn3-8 and their expression profiles were taken together in the same figure.

Three general expression profiles could be distinguished for the three structurally different classes described in this report. Psdhn1 expression showed minor fluctuations. However slightly elevated mRNA levels could be observed in the middle of the experimental period for both provenances. Psdhn2 mRNA levels decreased over time. Apart from an apparent dip in RNA levels at week 41, levels remained relatively constant and then dropped rapidly in the course of 4 weeks. Differences in timing of this drop were observed between the two provenances, which occurred earlier in apical buds of Lindås (from week 45 on) than in A70 (from week 49 on).

The opposite was found for the large group of dehydrins, Psdhn3-8. The mRNA levels of this group increased during the experimental period. In apical buds of A70 a sharp increase is observed in week 43 and in Lindås in week 45. The increment in mRNA levels of this group of genes is larger for A70 compared to Lindås. During the rest of the experimental period these mRNA levels remained high and relatively constant.

Microarray and RT-PCR results were in agreement. RT-PCR data for Lindås were shown in FIG. 3 and additional timepoints were included in order to match the dataset obtained for A70. In the RT-PCR, primers were used which amplify all six members of the Psdhn3-8 group simultaneously in order to maintain the same level of specificity as in microarray hybridisations.

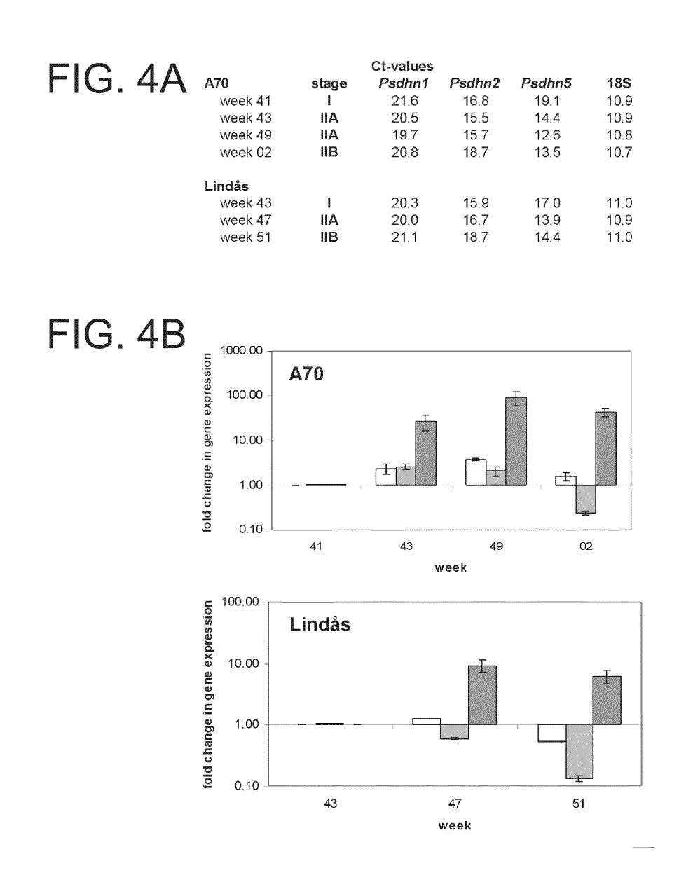

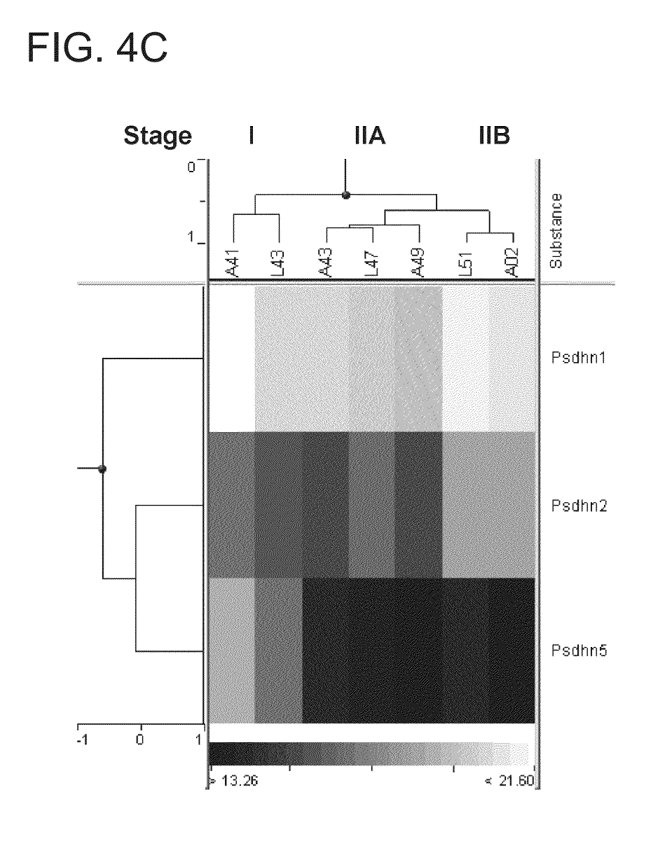

In FIGS. 4A-4C RT-PCR data are shown for Psdhn1 (SEQ ID No. 10), Psdhn2 (SEQ ID No. 1) and Psdhn5 (SEQ ID No. 2). The points are chosen in such a way as to represent the different stages with respect to the expression profiles of the different dehydrins. In this case a specific primer set for Psdhn5 was used. Relatively high numbers of cDNA clones were present for this gene in the cDNA libraries (Table 1).

High C_{T}-values were measured for both provenances using Psdhn1 primers reflecting the relative low abundance of the corresponding mRNA during the whole experimental period. In stage I, higher C_{T}-values for Psdhn5 were found for provenance A70 compared to Lindås, which points to lower mRNA levels. The difference between the provenances disappeared after the rise in mRNA levels in stage II. Low C_{T}-values found with Psdhn5 primers in this stage indicate high mRNA levels.

FIG. 4B shows that within stage II one can discriminate between early (IIA) and late (IIB) stages. Psdhn2 contributes mainly to that difference. Dehydrin C_{T}-values were used for hierarchical clustering and the result of it is shown in FIG. 4C. This visualises clearly the different stages.

In conclusion, differential expression of structurally divergent dehydrins as shown in this study indicates specialised functions during autumn and winter in apical buds. Although frost tolerance and dormancy develop simultaneously in Pinus sylvestris seedlings, frost tolerance seems to be the most consistent feature. Growth cessation always takes place but true rest (endodormancy in the terminology of Lang 1987) is not always reached (Dormling 1993). Dormancy, measured by monitoring days-to-bud-break of seedlings transferred at various timepoints to optimal growing conditions, did not show significant variation during the period examined. The results from the frost tolerance measurements however, are clear (FIGS. 2A-2B). It is known from studies using plants from different origins that northern provenances develop frost tolerance earlier than plants from southern origins (Dormling 1993, Repo et al. 2000) even when grown under identical conditions (Perks and McKay 1997). Latitudinal origin accounts for the lower freeze induced Shoot Electrolyte Values (higher frost tolerance) at week 39 for provenance Lindås. The fact that we examined one-year-old seedlings, which haven't experienced a winter before, points to a heritable trait. Comparison of the development of frost tolerance with the experienced cold, measured as the Cold Index (FIGS. 2A-2B), indicates that hardening is complete before a sharp rise in Cold Index. This again confirms that hardening is a highly regulated process that is triggered by genetic and environmental factors exerting their influence before real harsh conditions occur.

During the process of hardening, different structural types of dehydrin play different roles. As was already reported by Kontunen and Laine (2001), dehydrin protein can be detected throughout the season but certain dehydrin proteins were mainly detected during the growing season or during winter.

The current inventors established that during autumn and winter the mRNA level of SK₄ type Psdhn1 changes to some extent. This dehydrin may contribute to a certain base level of resistance to dehydration or has a function in protecting specific cellular structures. Abundance of SK₂ type Psdhn2 however is high in autumn, when hardening develops, and decreased during winter. A large group of K₂ type dehydrin genes, Psdhn3-8, showed increasing mRNA levels in autumn, before maximal levels of frost tolerance were reached. Realtime RT-PCR was used to obtain data specific for all dehydrin types and in FIGS. 4A-4C data are presented for Psdhn5. The expression profile of this dehydrin gene matched the profile obtained from the microarray.

In summary, high expression of the SK₂ type Psdhn2 is found when hardening proceeds. Subsequently, increase of K₂ type Psdhn3-8 mRNA levels coincide with high levels of frost tolerance. In comparison with Lindås, the rise in Psdhn3-8 mRNAs found in A70 is earlier and the increment is larger (FIG. 3). This group of dehydrins contributes to high levels of frost tolerance, resulting from the fact that A70 needs a steeper rise in frost tolerance to reach the same level as Lindås at week 43. Low initial mRNA levels of Psdhn3-8 are associated with the low level of frost tolerance found in shoots of A70 pines. The most indicative parameter for induction of hardening, dormancy and in particular stress and/or cold tolerance is therefore the ratio between SK and K type dehydrins, shifting from high SK₄, SK₂ and relatively low K₂, to a lower SK₄, SK₂ and a higher K₂ level.

Hence, with respect to dehydrin expression, different phases can be recognised during autumn and winter. This is evidenced by exploring various calculation methods using RT-PCR data. The results of this are shown in FIGS. 4A-4C. These data show that the expression pattern of the specified dehydrin Psdhn5, equals that of the general pattern of Psdhn 3, 4, 6, 7, 8 (SEQ ID No's 5, 6, 7, 8 and 9 respectively) found using the microarray.

The clearest distinction between the phases can be obtained using hierarchical clustering. Here it is evident that even the minor variation in expression of the SK₄ Psdhn1 gene does contribute to the stage definition. When these figures are combined with the frost tolerance data from FIGS. 2A-2B, a general observation is that when stage II is entered, frost tolerance is reaching its maximal value soon. When this pattern is established for different provenances or different offspring in a crossing/breeding experiment, this set of indicator genes may be applied according to the method of this invention to calculate a ratio between SK-type and K-type dehydrins. This will be most usefull for a rapid stage determination in nursery- and plantbreeding practice, in particular for Pinaceae and for forestry purposes. Determining the level of stress tolerance will also be most suitable for testing Pinaceae seedlings in the autumn, before they are taken from the field and stored till spring in a climatised winter storage at approximately −2° C. to +4° C. The level of hardening and hence the optimal timing for storage is determined by the method according to the invention. The ratios will be usefull as indicators for different stages of hardening or dormancy and the resulting stress tolerance, in particular frost tolerance and winter hardiness and re-growth potential in spring, for instance after winter storage.

### REFERENCES

Aharoni, A., L. C. Keizer, H. J. Bouwmeester, Z. Sun, M. Alvarez Huerta, H. A. Verhoeven, J. Blaas, A. M. van Houwelingen, R. C. de Vos, H. van der Voet, R. C. Jansen, M. Guis, J. Mol, R. W. Davis, M. Schena, A. J. van Tunen, A. P. O'Connell. 2000. Identification of the SAAT gene involved in strawberry flavor biogenesis by use of DNA microarrays. Plant Cell 12(5):647-662.
Aart van Amerongen and Marjo Koets (2005) Simple and rapid bacterial protein and DNA diagnostic methods based on signal generation with colloidal carbon particles. In: Rapid methods for biological and chemical contaminants in food and feed. Eds. A. van Amerongen, D. Barug and M. Lauwaars, Wageningen Academic Publishers, Wageningen, The Netherlands, ISBN: 9076998531, pages 105-126.
Alsheikh, M. K., B. J. Heyen and S. K. Randall. 2003. Ion binding properties of the dehydrin ERD14 are dependent upon phosphorylation. J. Biol. Chem. 278(42):40882-40889.
Artlip, T. S., A. M. Callahan, C. L. Basset and M. E. Wisniewski. 1997. Seasonal expression of a dehydrin gene in sibling deciduous and evergreen genotypes of peach (Prunus persica (L.) Batsch). Plant Mol. Biol. 33:61-70.
Bigras, F. J. 1996. Conifer bud dormancy and stress resistance: A forestry perspective. In Plant dormancy. Physiology, Biochemistry and Molecular Biology. Ed. G. A. Lang. CAB International, Oxon UK, pp 171-192.
Campbell, S. A. and T. J. Close. 1997. Dehydrins: genes, proteins, and associations with phenotypic traits. New Phytol. 137:61-74.
Chang, S., J. Puryear and J. Cairney. 1993. A simple and efficient method for isolating RNA from pine trees. Plant Mol. Biol. Rep. 11(2):113-116.
Choi, D. W., B. Zhu and T. J. Close. 1999. The barley (Hordeum vulgare L.) dehydrin multigene family: sequences, allele types, chromosome assignments, and expression characteristics of 11 Dhn genes of cv Dicktoo. Theor. Appl. Genet. 98:1234-1247.
Christersson, L. 1978. The influence of photoperiod and temperature on the development of frost hardiness in seedlings of Pinus sylvestris and Picea abies. Physiol. Plant. 44:288-294.
Close, T. J., R. D. Fenton and F. Moonan. 1993. A view of plant dehydrins using antibodies specific to the carboxy terminal peptide. Plant Mol. Biol. 23:279-286.
Close, T. J. 1996. Dehydrins: Emergence of a biochemical role of a family of plant dehydration proteins. Phys. Plant. 97:795-803.
Close, T. J. 1997. Dehydrins: A commonalty in the response of plant to dehydration and low temperature. Phys. Plant. 100:291-296.
Doom, W. G., P. A. Balk, A. M. van Houwelingen, F. A. Hoeberichts, R. D. Hall, O. Vorst, C. van der Schoot and M. F. van Wordragen. 2003. Gene expression during anthesis and senescence in Iris flowers. Plant. Mol. Biol. 53:845-863.
Dormling I. 1993. Bud dormancy, frost hardiness, and frost drought in seedlings of Pinus sylvestris and Picea abies. In Advances in plant cold hardiness. Ed. Paul H. Li and Lars Christersson. CRC Press, Florida, pp 285-298.
Ekberg I., G. Eriksson and I. Dormling. 1979. Photoperiodic reactions in conifer species. Holarctic Ecology 2:255-263.
Heyen. B. J., M. K. Alsheikh, E. A. Smith, C. F. Torvik, D. F. Seals and S. K. Randall. 2002. The Calcium-binding activity of a vacuole-associated, dehydrin-like protein is regulated by phosphorylation. Plant Physiol. 130:675-687.
Jarvis, S. B., M. A. Taylor, M. R. Macleod and H. V. Davies. 1996. Cloning and characterisation of the cDNA clones of three genes that are differentially expressed during dormancy-breakage in the seeds of Douglas fir (Pseudotsuga menzeisii). J. Plant Physiol. 147:559-566.
Karlson D. T., T. Fujino, S. Kimura, K. Baba, T. Itoh and E. N. Ashworth. 2003. Novel plasmodesmata association of dehydrin-like proteins in cold acclimated red-osier dogwood (Cornus sericea). Tree Physiol. 23:759-767.
Koag, M., R. D. Fenton, S. Wilkens and T. J. Close. 2003. The binding of Maize DHN1 to lipid vesicles. Gain of structure and lipid specificity. Plant Physiol. 131:309-316.
Marjo Koets, Nathalie Barbier, Emil Wolbert, Hans Mooibroek and Aart van Amerongen (2003) Rapid and simple one-step and mini-array methods to detect and quantify amplified DNA and proteins using ligand-labeled colloidal particles. In: Proceedings EURO FOOD CHEM XII—Strategies for Safe Food, 24-26 Sep. 2003, Brugge, Belgium, pages 121-124.
Kontunen-Soppela, S. and K. Laine. 2001. Seasonal fluctuation of dehydrins is related to osmotic status in Scots pine needles. Trees 15:425-430.
Diane Kozwich, Kristine A. Johansen, Keli Landau, Christopher A. Roehl, Sam Woronoff, and Patrick A. Roehl (2000) Development of a novel, rapid integrated Cryptosporidium parvum detection assay, Applied and Environmental Microbiology 66, 2711-2717. (Xtrana Inc., Denver, Colo. 80230)
Lang, G. A. 1987. Dormancy: a new universal terminology. HortScience 22:817-820.
Levi, A., G. R. Panta, C. M. Parmentier, M. M. Muthalif, R. Arora, S. Shanker and L. J. Rowland. 1999. Complementary DNA cloning, sequencing and expression of an unusual dehydrin from blueberry floral buds. Physiol. Plant. 107:98-109
Lim, C. C., S. L. Krebs and R. Arora. 1999. A 25-kDa dehydrin associated with genotype- and age-dependent leaf freezing-tolerance in Rhododendron: a genetic marker for cold hardiness? Theor. Appl. Genet. 99:912-920.
Lindström, A. and L. Häkansson. 1996. EC-metoden—et sätt at bestämma skogplantors lagringsbarhet. Sveriges Lantbruksuniversitet, Institutionen för skogsproduktion. Stencil nr. 95, 30 pp.
Livak K. J. and T. D. Schmittgen. 2001. Analysis of relative gene expression data using real-time quantitative PCR and the 2^{-ΔΔc}_{T} method. Methods 25:402-408.
Momma, M., S. Kaneko, K. Haraguchi and U. Matsukura. (2003) Peptide mapping and assessment of cryoprotective activity of 26/27-kDa dehydrin from soybean seeds. Biosci. Biotechnol. Biochem. 67(8):1832-1835.
McKay, H. M. 1992. Electrolyte leakage from fine roots of conifer seedlings: a rapid index of plant vitality following cold storage. Can. J. For. Res. 22: 1371-1377.
Nylander, M., J. Svensson, E. T. Palva and B. V. Welin. 2001. Stress-induced accumulation and tissue-specific localization of dehydrins in Arabidopsis thaliana. Plant Mol. Biol. 45:263-279.
Perks, M. P. and H. M. Mckay. 1997. Morphological and physiological differences in Scots pine seedlings of six different origins. Forestry 70(3):223-232.
Porat, R., D. Pavoncello, S. Lurie and T. G. Mccollum. 2002. Identification of a grapefruit cDNA belonging to a unique class of citrus dehydrins and characterization of its expression patterns under temperature stress conditions. Physiol. Plant. 115:598-603.
Repo, T., G. Zhang, A. Ryyppö, R. Rikala and M. Vuorinen. 2000. The relation between growth cessation and frost hardening in Scots pins of different origins. Trees 14:456-464.
Richard, S., M. Morency, C. Drevet, L. Jouanin and A. Seguin. 2000. Isolation and characterization of a dehydrin gene from white spruce induced upon wounding, drought and cold stress. Plant Mol. Biol. 43:1-10.
Rinne, P. L. H., P. L. M. Kaikuranta, L. H. W. van der Plas and C. van der Schoot. 1999. Dehydrins in cold-acclimated apices of birch (Betula pubescens Ehrh.): production, localization and potential role in rescuing enzyme function during dehydration. Planta 209:377-388.
Rowland L. J. and R. Arora. 1997. Proteins related to endodormancy (rest) in woody perennials. Plant Science 126:119-144.
Stattin, E., Hellqvist, C. and A. Lindström. 2000. Storability and root freezing tolerance of Norway spruce (Picea abies) seedlings. Can. J. For. Res. 30:964-970.
Wang, W., D. Pelah, T. Alergand, O. Shoseyov and A. Altman. 2002. Characterization of SP1, a stress-responsive, boiling soluble, homo-oligomeric protein from aspen. Plant Physiology 130:865-875.
Welling, A., T. Moritz, E. T. Palva and O. Junttila. 2002. Independent activation of cold acclimation by low temperature and short photoperiod in hybrid aspen. Plant Physiology 129:1633-1641.
Welling, A., P. Rinne, A. Viherä-Aarnio, S. Kontunen-Soppela, P. Heino and E. T. Palva. 2004. Photoperiod and temperature differentially regulate the expression of two dehydrin genes during overwintering of birch (Betula pubescens Ehrh.). J. Exp. Botany 55 (396):507-516.
Wise, M. J., A. Tunnacliffe. 2004. POPP the question: what do LEA proteins do? Trends in Plant Science 9(1):13-17.
Zhu, B., D. W. Choi, R. Fenton and T. J. Close. 2000. Expression of the barley dehydrin multigene family and the development of freezing tolerance. Mol. Gen. Genet. 264:145-153

1.-15. (canceled)

16. A method for selecting a Pinaceae plant for use in a breeding program, comprising the steps of:
(a) obtaining a first sample of a Pinaceae plant during or following a period of hardening of said plant;
(b) obtaining a second sample of said Pinaceae plant during or following a period of dormancy;
(c) measuring the level of a SK-type dehydrin messenger ribonucleic acid (mRNA) or protein and the level of a K-type dehydrin messenger ribonucleic acid (mRNA) or protein in said first sample;
(d) measuring the level of a SK-type dehydrin messenger ribonucleic acid (mRNA) or protein and the level of a K-type dehydrin messenger ribonucleic acid (mRNA) or protein in said second sample;
(e) comparing the level of said SK-type dehydrin messenger ribonucleic acid (mRNA) or protein and the level of said K-type dehydrin messenger ribonucleic acid (mRNA) or protein in said first sample to the level of said SK-type dehydrin messenger ribonucleic acid (mRNA) or protein and the level of said K-type dehydrin messenger ribonucleic acid (mRNA) or protein in said second sample;
wherein said Pinaceae plant is selected for use in said breeding program if said Pinaceae plant exhibits a decreased level of the SK-type messenger ribonucleic acid (mRNA) or protein and an increased level of the K-type messenger ribonucleic acid (mRNA) or protein in said second sample as compared to said first sample.

17. The method of claim 16, comprising obtaining said second sample from 1 to 8 weeks after obtaining said first sample.

18. The method of claim 16, wherein said SK-type dehydrin messenger ribonucleic acid (mRNA) or protein is a SK₄ dehydrin messenger ribonucleic acid (mRNA) or protein or a SK₂ dehydrin messenger ribonucleic acid (mRNA) or protein.

19. The method of claim 18, wherein said SK₂ dehydrin messenger ribonucleic acid (mRNA) has at least 85, 90, 95, 98 or 99% identity with a Psdhn2 as set forth in SEQ ID No. 1.

20. The method of claim 16, wherein said K-type dehydrin messenger ribonucleic acid (mRNA) or protein is a K₂-type dehydrin messenger ribonucleic acid (mRNA) or protein.

21. The method of claim 20, wherein said K₂-type dehydrin messenger ribonucleic acid (mRNA) has at least 85, 90, 95, 98 or 99% identity with a Psdhn5 as set forth in SEQ ID No. 2.

22. The method of claim 16, wherein said Pinaceae plant is selected from the genus consisting of Pinus, Picea, Pseudotsuga, Tsuga, Larix, Abies, and Cedrus.

23. The method of claim 16, wherein said measuring in steps (c) and (d) comprises the quantitative amplification of a SK-type dehydrin messenger ribonucleic acid (mRNA) and a K-type dehydrin messenger ribonucleic acid (mRNA) obtained from said sample or a complementary deoxyribonucleic acid (cDNA) obtained from said first sample and said second sample.

24. The method of claim 16, wherein said sample is a leaf, a flower, a root, a shoot, a twig, a fruit, pollen, a seed, an embryo, a seedling, a bud, a plant cell or a callus.

25. The method of claim 24, wherein said bud is an apical bud.

26. A method of identifying a Pinaceae plant which is cold stress tolerant or drought stress tolerant, comprising the steps of:
a. obtaining a first sample of a Pinaceae plant during or following a period of hardening of said plant;
b. obtaining a second sample of said Pinaceae plant during or following a period of cold;
c. measuring the level of a SK-type dehydrin messenger ribonucleic acid (mRNA) or protein and the level of a K-type dehydrin messenger ribonucleic acid (mRNA) or protein in said first sample;
d. measuring the level of a SK-type dehydrin messenger ribonucleic acid (mRNA) or protein and the level of a K-type dehydrin messenger ribonucleic acid (mRNA) or protein in said second sample;
e. comparing the level of said SK-type dehydrin messenger ribonucleic acid (mRNA) or protein and the level of said K-type dehydrin messenger ribonucleic acid (mRNA) or protein in said first sample to the level of said SK-type dehydrin messenger ribonucleic acid (mRNA) or protein and the level of said K-type dehydrin messenger ribonucleic acid (mRNA) or protein in said second sample;
wherein said Pinaceae plant is identified as cold stress tolerant or drought stress tolerant if said Pinaceae plant exhibits a decreased level of the SK-type messenger ribonucleic acid (mRNA) or protein and an increased level of the K-type messenger ribonucleic acid (mRNA) or protein in said second sample as compared to said first sample.

27. The method of claim 26, comprising obtaining said second sample from 1 to 8 weeks after obtaining said first sample.

28. The method of claim 26, wherein said SK-type dehydrin messenger ribonucleic acid (mRNA) or protein is a SK₄ dehydrin messenger ribonucleic acid (mRNA) or protein or a SK₂ dehydrin messenger ribonucleic acid (mRNA) or protein.

29. The method of claim 28, wherein said SK₂ dehydrin messenger ribonucleic acid (mRNA) has at least 85, 90, 95, 98 or 99% identity with a Psdhn2 as set forth in SEQ ID No. 1.

30. The method of claim 26, wherein said K-type dehydrin messenger ribonucleic acid (mRNA) is a K₂-type dehydrin messenger ribonucleic acid (mRNA) or protein.

31. The method of claim 30, wherein said K₂-type dehydrin messenger ribonucleic acid (mRNA) has at least 85, 90, 95, 98 or 99% identity with a Psdhn5 as set forth in SEQ ID No. 2.

32. The method of claim 26, wherein said Pinaceae plant is selected from the genus consisting of Pinus, Picea, Pseudotsuga, Tsuga, Larix, Abies, and Cedrus.

33. The method of claim 26, wherein said measuring in steps (c) and (d) comprises the quantitative amplification of a SK-type dehydrin messenger ribonucleic acid (mRNA) and a K-type dehydrin messenger ribonucleic acid (mRNA) obtained from said sample or a complementary deoxyribonucleic acid (cDNA) obtained from said first sample and said second sample.

34. The method of claim 26, wherein said sample is a leaf, a flower, a root, a shoot, a twig, a fruit, pollen, a seed, an embryo, a seedling, a bud, a plant cell or a callus.

35. The method of claim 26, wherein said method comprises the use of a Polymerase Chain Reaction (PCR) technique.
